(19) United States
(12) Patent Application Publication
(10) Pub. No.: US 2024/0197727 A1
(43) Pub. Date: Jun. 20, 2024

(54) SOLID DISPERSIONS COMPRISING AMORPHOUS PIMOBENDAN AND ONE OR MORE STABILIZING POLYMERS

(71) Applicant: Boehringer Ingelheim Vetmedica GmbH, Ingelhelm am Rhein (DE)

(72) Inventors: Péter LACZAY, Budapest (HU); Péter Lajos Sóti, Tiszakécske (HU)

(21) Appl. No.: 18/537,432

(22) Filed: Dec. 12, 2023

(30) Foreign Application Priority Data
Dec. 15, 2022 (EP) 22213699.6

(51) Int. Cl. A61K 31/501; A61K 9/20

(52) U.S. Cl. CPC A61K 31/501; A61K 9/2009; A61K 9/2013; A61K 9/2018; A61K 9/2054; A61K 9/2059; A61K 9/2068

(57) ABSTRACT

The invention relates to novel solid dispersions comprising amorphous pimobendan and one or more stabilizing polymers as well as processes of manufacturing thereof and corresponding pharmaceutical compositions.

### INCORPORATION BY REFERENCE

All references cited herein, are incorporated by reference herein, in their entirety.

### FIELD OF THE INVENTION

The invention relates to the field of medicine, particularly veterinary medicine. In particular, the invention relates to novel solid dispersions comprising amorphous pimobendan and one or more stabilizing polymers and processes of manufacturing thereof as well as corresponding pharmaceutical compositions.

### BACKGROUND OF THE INVENTION

Pimobendan (4,5-dihydro-6-(2-(4-methoxyphenyl)-1H-benzimidazol-5-yl)-5-methyl-3(2H))-pyridazinone) is a benzimidazole pyridazone derivative, which was described in EP 0 008 391 as a substance having cardiotonic, hypotensive and antithrombotic activities.

Pimobendan is known to exhibit polymorphia. Over ten potential polymorphs and solvatomorphs of chiral pimobendan were described (Rekis at al., 2018).

EP 0 439 030 discloses the low solubility of pimobendan in aqueous environment, which is characterized by a highly pH-dependent nature. Depending on the buffer system used, about 100 to 300 mg/liter dissolve at a pH between 1 and 3, but at pH 5 only about 1 mg/liter will dissolve in water. In humans, this phenomenon resulted in strongly fluctuating blood concentrations, which levels were often too low. These unsatisfactory absorption characteristics were explained by the high pH-dependency of the solubility of pimobendan in aqueous media and by fluctuating pH conditions in the gastrointestinal tract of the test subjects. According to this patent publication, the low solubility and high pH dependency of the solubility of pimobendan can be overcome by using an intimate dry admixture of powdered pimobendan and powdered citric acid wherein said admixture is up to about one part by weight of pimobendan per no less than about five parts by weight of citric acid and pharmaceutically active carriers, being filled into capsules or compressed into tablets for oral administration. The strongly fluctuating blood concentrations are said to be prevented by the acid microsphere, which is caused by the dissolving rate of citric acid, formed around the pimobendan particles. Said microsphere is always acidic and ensures a reliable, practically 30 pH-independent dissolution and absorption of pimobendan.

WO 2005/084647 relates to a novel solid formulation comprising pimobendan, which is homogeneously dispersed in a polyvalent acid selected from the group consisting of citric acid, acetic acid, maleic acid, tartaric acid or its anhydride, and a flavouring substance. According to said publication, the high quantity of citric acid and the acidic taste of it is not readily accepted by most animals. Thus, these formulations have to be force-fed to the animals or mixed with food prior to application. According to this patent publication, these difficulties can be overcome by using this novel formulation preferably in the form of tablets. Most preferred is a tablet characterized in that the tablet comprises 1.25 mg, 2.5 mg, 5 mg or 10 mg pimobendan, and further comprises citric acid, preferably at an amount of 50 mg/g of the solid formulation, artificial beef flavour and pharmaceutically acceptable excipients. WO 2008/055871 relates to a liquid preparation comprising an etherified cyclodextrin derivative and a pimobendan or a pharmaceutical acceptable salt thereof.

WO 2010/010257 relates to the use of a coating composition for application to a solid veterinary pharmaceutical composition made from pimobendan by a method of film coating comprising a powder appetizing material, a binder and a solvent.

WO 2010/055119 discloses a novel formulation, comprising pimobendan and an organic carboxylic acid, wherein the only organic carboxylic acid is succinic acid, and the weight ratio of succinic acid to pimobendan is at least 11:1.

EP 2 338 493 provides new crystalline forms of pimobendan, the solubility characteristics of which are such that adding an organic acid or an anhydride thereof is not needed for ensuring a satisfactory resorption of the substance. WO 2015/082389 is directed to a composition comprising particles of pimobendan with an integral coating of a carrier matrix, which serve to ensure a rapid dissolution of the active substance at each pH condition representing the gastrointestinal tract and therefore a reliable absorption, and a method of pimobendan microencapsulation using the spray congealing technology and incorporating the coated particles into oral formulations, for example into tablets.

WO 2017/103054 discloses a solid formulation, comprising pimobendan or a pharmaceutically acceptable salt thereof, which is dispersed in malic acid, and a flavor suitable for small animals. It also relates to a wet granulation process for preparation of the formulation.

WO 2021/081366 is related to a chewable oral formulation containing pimobendan in granular form prepared by mixing it with lactose and/or dicalcium phosphate, granulating and coating the mixture with a polyvinyl alcohol-polyethylene glycol graft copolymer.

Further references are as follows:

CN 112 618 505 A describes a compound pharmaceutical composition containing benazepril and pimobendan for pets and a preparation method thereof.

WO 2011/042463 describes a pharmaceutical composition comprising at least one hyperbranched polymer and at least one pharmaceutically active ingredient, wherein the polymer and the pharmaceutically active ingredient are present in a specific weight ratio, and to a process for the preparation of said pharmaceutical composition. Vasconcelos T et al., Drug Discovery Today 2012, 12: 1068-1075, describe solid dispersions as a strategy to improve oral bioavailability for poorly water-soluble drugs.

The objective underlying the present invention is to provide an improved pimobendan formulation, which overcomes the problems of the prior art.

### SUMMARY OF THE INVENTION

Surprisingly, the inventors have succeeded in overcoming the low and highly pH-dependent solubility of pimobendan and ensuring a very satisfactory dissolution and transmembrane flux of pimobendan at different biorelevant conditions by forming a solid dispersion of amorphous pimobendan in one or more stabilizing polymers. In the solid dispersion pimobendan is present in a substantially amorphous form.

In one aspect, the objective of the present invention has therefore surprisingly been solved by providing a solid dispersion comprising, preferably consisting of, amorphous pimobendan, preferably substantially amorphous pimobendan, and one or more stabilizing polymers.

In another aspect, the objective of the present invention has therefore surprisingly been solved by providing a solid dispersion comprising amorphous pimobendan and one or more stabilizing polymers.

In another aspect, the objective of the present invention has therefore surprisingly been solved by providing a solid dispersion consisting of amorphous pimobendan and one or more stabilizing polymers.

In another aspect, the objective of the present invention has therefore surprisingly been solved by providing a solid dispersion comprising substantially amorphous pimobendan and one or more stabilizing polymers.

In another aspect, the objective of the present invention has therefore surprisingly been solved by providing a solid dispersion consisting of substantially amorphous pimobendan and one or more stabilizing polymers.

In yet another aspect, the objective of the present invention has surprisingly been solved by providing a process of preparing the solid dispersion as herein disclosed and/or claimed, wherein the solid dispersion is prepared by a melting-based process, preferably by hot melt extrusion, or a solvent evaporation-based process, preferably by spray drying.

In yet another aspect, the objective of the present invention has therefore surprisingly been solved by providing a solid dispersion comprising, preferably consisting of, amorphous pimobendan, preferably substantially amorphous pimobendan, and one or more stabilizing polymers obtainable by the processes as herein disclosed and/or claimed.

In yet another aspect, the objective of the present invention has surprisingly been solved by providing a solid dispersion comprising amorphous pimobendan and one or more stabilizing polymers obtainable by the processes as herein disclosed and/or claimed.

In yet another aspect, the objective of the present invention has surprisingly been solved by providing a solid dispersion comprising substantially amorphous pimobendan and one or more stabilizing polymers obtainable by the processes as herein disclosed and/or claimed.

In yet another aspect, the objective of the present invention has surprisingly been solved by providing a solid dispersion consisting of amorphous pimobendan and one or more stabilizing polymers obtainable by the processes as herein disclosed and/or claimed.

In yet another aspect, the objective of the present invention has surprisingly been solved by providing a solid dispersion consisting of substantially amorphous pimobendan and one or more stabilizing polymers obtainable by the processes as herein disclosed and/or claimed.

In yet another aspect, the objective of the present invention has surprisingly been solved by providing a pharmaceutical composition comprising the solid dispersion as herein disclosed and/or claimed and one or more pharmaceutically acceptable excipients, optionally additionally comprising crystalline form(s) of pimobendan (preferably according to FIG. 1), wherein preferably the pharmaceutical composition is a tablet, more preferably a chewable tablet.

The amorphous state of pimobendan in the solid dispersion is critical for increasing its solubility. With the drug substance in the amorphous form, no energy is required to break the drug crystal lattice. For this reason, relative to the crystalline form(s), the amorphous form of pimobendan was found to achieve substantially higher apparent solubility and markedly faster and higher dissolution at different biorelevant conditions that characterize the gastrointestinal tract of dogs. The solid dispersion of amorphous pimobendan according to the present invention was found to result in higher transmembrane flux due to a higher supersaturation, which can improve the bioavailability of the drug substance.

### BRIEF DESCRIPTION OF THE DRAWINGS

FIG. 1: XRPD diffractograms of crystalline pimobendan (AGC Pharma Chemicals, Spain) and the solid dispersions comprising substantially amorphous pimobendan prepared according to Examples 1 and 4.

FIG. 2: In vitro dissolution profiles of the pharmaceutical composition according to Example 5 and a commercially available pimobendan tablet formulation at pH 3.0 and pH 6.5, respectively. Conditions of testing: instrument similar to USP apparatus 2, V=1000 ml, rotation speed=50 rpm, chromatographic instrument: Agilent Infinity 1290 UHPLC, RP18, 50×3.0 mm, 1.7 pm column.

FIG. 3: In vitro biorelevant volume dissolution and permeation of the pharmaceutical composition according to Example 5 and a commercially available pimobendan tablet formulation at default pH conditions characterizing the stomach (pH 3.0) and the small intestine (pH 6.2) by using simulated gastric and intestinal fluids for fasted dogs (Biorelevant, London, United Kingdom). Concentration of the drug substance was determined by immersed UV probes.

FIG. 4: In vitro biorelevant volume dissolution and permeation of the pharmaceutical composition according to Example 5 and a commercially available pimobendan tablet formulation at high pH conditions characterizing the stomach (pH 6.5) and the small intestine (pH 7.5) by using simulated gastric and intestinal fluids for fasted dogs (Biorelevant, London, United Kingdom). Drug substance concentration was determined by immersed UV probes.

FIG. 5: XRPD diffractograms of the solid dispersions comprising substantially amorphous pimobendan prepared according to Example 2.

FIG. 6: XRPD diffractograms of the solid dispersions comprising substantially amorphous pimobendan prepared according to Example 3.

### DETAILED DESCRIPTION OF THE INVENTION

Before the embodiments of the present invention are described in further details it shall be noted that as used herein and in the appended claims, the singular forms “a”, “an”, and “the” include plural reference unless the context clearly dictates otherwise.

Unless defined otherwise, all technical and scientific terms used herein have the same meanings as commonly understood by one of ordinary skill in the art to which this invention belongs. All given ranges and values may vary by 1 to 5% unless indicated otherwise or known otherwise by the person skilled in the art, therefore, the term “about” was usually omitted from the description and claims. Although any methods and materials similar or equivalent to those described herein can be used in the practice or testing of the present invention, the preferred methods, devices, and materials are now described. All publications mentioned herein are incorporated herein by reference for the purpose of describing and disclosing the substances, excipients, carriers, and methodologies as reported in the publications which might be used in connection with the invention. Nothing herein is to be construed as an admission that the invention is not entitled to antedate such disclosure by virtue of prior invention.

The term “drug” or “drug substance” as used herein refers to pimobendan (4,5-dihydro-6-(2-(4-methoxyphenyl)-1H-benzimidazol-5-yl)-5-methyl-3(2H))pyridazinone).

The term “solid dispersion” refers to a system in a solid state comprising at least two components, wherein one component is dispersed throughout the other component or components.

The term “solid dispersion” in connection with “amorphous pimobendan” as used herein refers to stable solid dispersions comprising amorphous pimobendan and one or more stabilizing polymers.

By the term “amorphous pimobendan” it is meant that the solid dispersion contains pimobendan in a substantially amorphous solid-state form.

A solid that is in the “amorphous” solid state form means that it is in a non-crystalline state. The solid-state form of a solid, such as pimobendan in the solid dispersion, may be determined by X-Ray Powder Diffraction (XPRD), Differential Scanning Calorimetry (DSC), or other standard techniques known to those of skill in the art.

The term “substantially amorphous pimobendan” or “pimobendan in a substantially amorphous solid-state form” means that no crystalline form(s) of pimobendan can be detected in the material/solid dispersion by XRPD and/or DSC, for example and preferably using the conditions described in the working examples herein.

The term “stabilizing polymer” as herein disclosed and/or claimed refers to any one of the hydrophilic polymers known to those of skill in the art, preferably to polymerization products of N-vinylpyrrolidone and vinylpyrrolidone-vinyl acetate, in particular polymerization products of N-vinylpyrrolidone, vinyl acetate and vinylpyrrolidone mixed with vinyl acetate; as well as polymeric methacrylates, more preferably to vinylpyrrolidone-vinyl acetate copolymer. The term shall also be understood to mean mixtures of any two or more of the aforementioned polymers.

The term “pharmaceutically acceptable excipients” includes all excipients that have been approved for use in human and/or veterinary medicinal products.

According to one embodiment, the solid dispersion as herein disclosed and/or claimed is provided, wherein the one or more stabilizing polymers is selected from the group consisting of: polymerization products of N-vinylpyrrolidone and vinylpyrrolidone-vinyl acetate; and/or polymerization products of N-vinylpyrrolidone, vinyl acetate and vinylpyrrolidone mixed with vinyl acetate; as well as polymeric methacrylates.

Polymerization products of N-vinylpyrrolidone and vinylpyrrolidone-vinyl acetate; and/or polymerization products of N-vinylpyrrolidone, vinyl acetate and vinylpyrrolidone mixed with vinyl acetate; primarily comprise povidone (polyvinylpyrrolidone), polyvinyl alcohol and copovidone [poly-(1-vinylpyrrolidone-co-vinylacetate)].

Polymeric methacrylates include the polymeric butyl 2-methylprop-2-enoate; polymeric 2-(dimethylamino)ethyl 2-mcthylprop-2-enoate; and polymeric methyl 2-methylprop-2-enoatc (e.g. EUDRAGIT® E portfolio from Evonik).

According to one embodiment, the solid dispersion as herein disclosed and/or claimed is provided, wherein the solid dispersion; and/or the one or more stabilizing polymers; and/or the polymerization products of N-vinylpyrrolidone and vinylpyrrolidone-vinyl acetate; and/or the polymerization products of N-vinylpyrrolidone, vinyl acetate and vinylpyrrolidone mixed with vinyl acetate; and/or polymeric methacrylates independently from each other do not comprise crospovidone (cross-linked polyvinylpyrrolidone, polyvinylpolypyrrolidone, PVPP). According to one embodiment, the solid dispersion as herein disclosed and/or claimed is provided, wherein the solid dispersion; and/or the one or more stabilizing polymers; and/or the polymerization products of N-vinylpyrrolidone and vinylpyrrolidone-vinyl acetate; and/or the polymerization products of N-vinylpyrrolidone, vinyl acetate and vinylpyrrolidone mixed with vinyl acetate; and/or polymeric methacrylates independently from each other do not comprise one or more hyperbranched polymers, optionally selected from the group consisting of: dendritic polymers, dendrimers, arborol, cascade, cauliflower or star polymers, polydisperse hyperbranched polymers and dendrigraft polymers, or other high molecular weight polymers which all have a specific branched structure containing a center atom or a molecule, which can be monomeric or polymeric, from where three or more chains emanate; hyperbranched polyimines, hyperbranched polyurethanes, hyperbranched polyamides, hyperbranched polyesteramines, hyperbranched polyesteramides, hyperbranched polymers comprising hydroxyl groups, ester groups, amido groups and/or carboxyl groups, and polyesteramide hyperbranched polymers, such as one or more hyperbranched polyesteramide having tertiary amine end groups and/or having hydroxyl end groups.

According to another embodiment, the solid dispersion as herein disclosed and/or claimed is provided, wherein the polymerization products of N-vinylpyrrolidone and vinylpyrrolidone-vinyl acetate; and/or the polymerization products of N-vinylpyrrolidone, vinyl acetate and vinylpyrrolidone mixed with vinyl acetate; are selected from the group consisting of: povidone (polyvinylpyrrolidone), polyvinyl alcohol and copovidone [poly-(1-vinylpyrrolidone-co-vinylacetate)]; and the polymeric methacrylates are selected from the group consisting of: polymeric butyl 2-methylprop-2-enoate; polymeric 2-(dunethylanino)ethyl 2-methylprop-2-enoate; and polymeric methyl 2-methylprop-2-enoate (e.g. EUDRAGIT® E portfolio from Evonik).

In one embodiment, the preferred stabilizing polymer to form solid dispersion of amorphous pimobendan is copovidone, which is more hydrophobic than povidone and provides higher protection against crystallization of the drug substance.

According to yet another embodiment, the solid dispersion as herein disclosed and/or claimed is provided, wherein the one or more stabilizing polymers is copovidone [poly-(1-vinylpyrrolidone-co-vinylacetate)], which is preferably the only stabilizing polymer in the solid dispersion.

According to yet another embodiment, the solid dispersion as herein disclosed and/or claimed is provided, wherein pimobendan is present in an amount of 1% to 80% by weight relative to the weight of the solid dispersion, preferably in an amount of 5% to 40% by weight relative to the weight of the solid dispersion and more preferably in an amount of 10% to 20% by weight relative to the weight of the solid dispersion.

According to yet another embodiment, the solid dispersion as herein disclosed and/or claimed is provided, wherein at least 70%, preferably 75%, more preferably 80%, even more preferably 85%, even more preferably 90%, even more preferably 91%, even more preferably 92%, even more preferably 93%, even more preferably 94%, even more preferably 95%, even more preferably 96%, even more preferably 97%, even more preferably 98%, even more preferably 99%, even more preferably 99.1%, even more preferably 99.2%, even more preferably 99.3%, even more preferably 99.4%, even more preferably 99.5%, even more preferably 99.6%, even more preferably 99.7%, even more preferably 99.8%, even more preferably 99.9%, even more preferably 99.95%, even more preferably 100% of the pimobendan is present in amorphous form.

The solid dispersion of amorphous pimobendan can be prepared by using a melting-based process or a solvent evaporation-based process. Preferably, the solid dispersion of amorphous pimobendan is prepared by hot melt extrusion. In general, extrusion is the process of changing the physical properties of the substance by forcing it through an orifice or die under controlled conditions. In pharmaceutical manufacturing, hot melt extrusion is the process of applying heat and pressure to melt a polymer matrix and disperse a drug substance at molecular level in it. Hot melt extrusion is a process well known to those skilled in the art for preparing solid dispersions. The solid dispersion of amorphous pimobendan according to the present invention can also be prepared by a solvent evaporation-based process such as spray drying. Spray drying is a process also well known to those skilled in the art for preparing solid dispersions. The solid dispersion of amorphous pimobendan can be formed by dispersing or dissolving the drug substance and the one or more stabilizing polymers in a suitable solvent to form a feed solution, pumping the feed solution through an atomizer into a drying chamber, and removing the solvent to form solid particles in the dying chamber. Examples of suitable solvents include dichlormethane, chloroform, ethanol, methanol, 2-propanol, ethyl acetate, acetone, water or mixtures thereof.

Although the solid dispersions of the present invention are preferably prepared using conventional hot melt extrusion or spray drying techniques, it will be understood that suitable solid dispersions may be formed utilizing other conventional techniques known to those skilled in the art, such as other melt-based processes or solvent removal processes.

According to one embodiment, the process being a hot melt extrusion process as herein disclosed and/or claimed is provided, comprising the steps of:
(a) processing pimobendan and the one or more stabilizing polymers by means of a pharmaceutical extruder, preferably a screw extruder, more preferably a twin-screw extruder, preferably at a barrel temperature of 120-200° C., more preferably at a barrel temperature of 150-180° C., to obtain an extrudate,
(b) comminuting the extrudate obtained in step (a) by using a granulator or pelletizer to obtain a granulate, and
(c) milling the granulate obtained in step (b) to obtain a solid dispersion, the solid dispersion preferably comprising particles with an average diameter of less than 500 μm.

According to another embodiment, the process being a spray drying process as herein disclosed and/or claimed is provided, comprising the steps of:
(a) dispersing or dissolving pimobendan and the one or more stabilizing polymers in one or more solvents, preferably selected from the group consisting of: dichloromethane, chloroform, ethanol, methanol, 2-propanol, ethyl acetate, acetone, water and mixtures thereof, to obtain a feed solution,
(b) pumping the feed solution obtained in step (a) through an atomizer into a drying chamber, and
(c) removing the one or more solvents in the drying chamber to obtain a solid dispersion, the solid dispersion preferably comprising particles with an average diameter of less than 500 μm.

According to one embodiment, the pharmaceutical composition as herein disclosed and/or claimed is provided, wherein the pharmaceutically acceptable excipients comprise at least one filler, at least one disintegrant, at least one lubricant and at least one flavor.

Pharmaceutically acceptable excipients can include one or more fillers, disintegrants, lubricants, glidants and flavors. Fillers include fillers known in the art such as cellulose, lactose, starch derivatives and mannitol. Preferred fillers am microcrystalline cellulose and lactose. Disintegrants include disintegrants known in the art including crospovidone (cross-linked polyvinylpyrolidone), croscarmellose, pregelatinized starch and sodium starch glycolate, the latter being preferred. Examples of lubricants are stearic acid, magnesium stearate, sodium stearyl fumarate. Preferred lubricants are magnesium stearate and talc. Examples of glidants are colloidal silicon dioxide and talc. A preferred glidant is talc. Flavors can include natural compounds such as dried yeast, meat or liver powders and synthetic aromas. Preferred flavors are pork liver powder and dried yeast.

Optionally, the pharmaceutical compositions of the present invention can also include crystalline forms of pimobendan to mitigate the supra-bioavailability of the amorphous drug substance, if needed. Due to the substantial, practically pH-independent dissolution and transmembrane flux of the amorphous drug substance its bioavailability can exceed that of the formulations prepared according to the state of the art.

The supra-bioavailability of the formulations comprising the drug substance substantially in amorphous state can be reduced by replacing a part of the amorphous drug substance with its crystalline form(s), if needed. Another option to mitigate the potential supra-bioavailability is to reduce the dose of the amorphous drug substance.

The pharmaceutical compostions can be prepared using a conventional process well known to those of skill in the art for preparing solid dosage forms.

In certain cases, mixing different amounts of crystalline pimobendan with (substantially) amorphous pimobendan may be done to adjust the bioavailability in the target animal (ensuring bioequivalence).

The solid dispersion of amorphous pimobendan prepared according to the present invention is processed with the pharmaceutically acceptable excipients and optionally the crystalline form(s) of the drug substance to obtain the pharmaceutical composition. Preferred dosage form is a chewable tablet. However, other conventional solid dosage forms, such as granules or soft chewable tablets, can also be prepared by using standard manufacturing methods known to those of skill in the art.

According to another embodiment, the pharmaceutical composition as herein disclosed and/or claimed is provided, further comprising pharmaceutically effective amounts of one or more further active ingredients. In one embodiment, the additional active ingredient may be angiotensin converting enzyme (ACE) inhibitors, aldosterone antagonists and/or loop diuretics.

According to yet another embodiment, the pharmaceutical composition as herein disclosed and/or claimed is provided, wherein the optional one or more further active ingredients selected from the group of angiotensin converting enzyme (ACE) inhibitors, aldosterone antagonists and/or loop diuretics are, independently from each other, benazepril, spironolactone, furosemide and/or derivatives thereof, in free form or in the form of a physiologically acceptable salt.

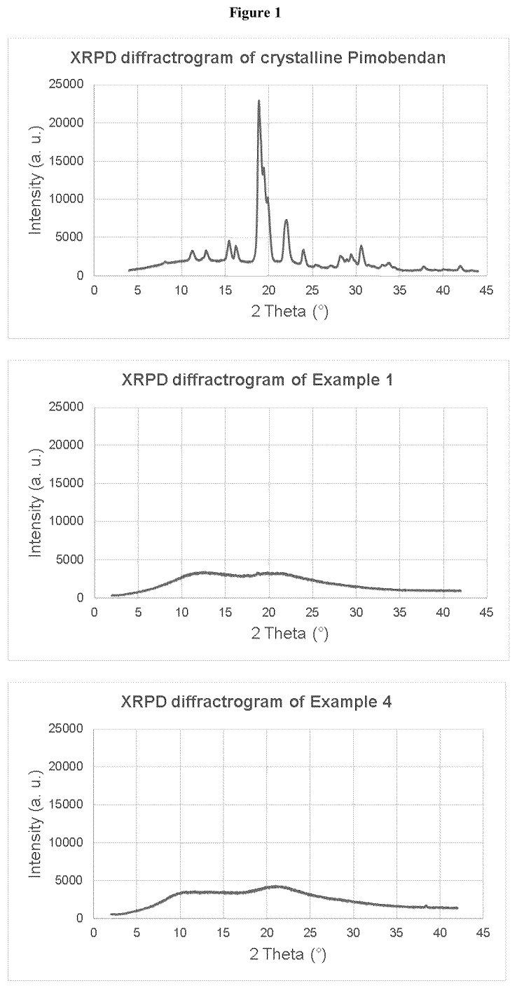

According to yet another embodiment, the pharmaceutical composition as herein disclosed and/or claimed is provided selected from the group consisting of: A, B, C, D and E (the crystalline pimobendan depicted in the tables below preferably refers to the crystalline pimobendan according to FIG. 1, AGC Pharma Chemicals, Spain):

| Components | Amounts (% w/w) |
|---|---|
| Solid dispersion of substantially amorphous | 6.3 |
| pimobendan and one or more stabilizing polymers | |
| Lactose monohydrate | 26.1 |
| Microcrystalline cellulose | 20.6 |
| Pork liver powder | 20.0 |
| Dried yeast | 10.0 |
| Starch, pregelatinized | 8.0 |
| Sodium starch glycolate | 6.0 |
| Talc | 2.0 |
| Magnesium stearate | 1.0 |

| Components | Amounts (% w/w) |
|---|---|
| Solid dispersion of substantially amorphous | 5.0 |
| pimobendan and one or more stabilizing polymers | |
| Lactose monohydrate | 27.4 |
| Microcrystalline cellulose | 20.6 |
| Pork liver powder | 20.0 |
| Dried yeast | 10.0 |
| Starch, pregelatinized | 8.0 |
| Sodium starch glycolate | 6.0 |
| Talc | 2.0 |
| Magnesium stearate | 1.0 |

| Components | Amounts (% w/w) |
|---|---|
| Solid dispersion of substantially amorphous | 3.1 |
| pimobendan and one or more stabilizing polymers | |
| Pimobendan crystalline | 0.3 |
| Lactose monohydrate | 29.0 |
| Microcrystalline cellulose | 20.6 |
| Pork liver powder | 20.0 |
| Dried yeast | 10.0 |
| Starch, pregelatinized | 8.0 |
| Sodium starch glycolate | 6.0 |
| Talc | 2.0 |
| Magnesium stearate | 1.0 |

| Components | Amounts (% w/w) |
|---|---|
| Solid dispersion of substantially amorphous | 3.1 |
| pimobendan and one or more stabilizing polymers | |
| Pimobendan crystalline | 0.3 |
| Lactose monohydrate | 30.0 |
| Microcrystalline cellulose | 29.6 |
| Pork liver powder | 20.0 |
| Starch, pregelatinized | 8.0 |
| Sodium starch glycolate | 6.0 |
| Talc | 2.0 |
| Magnesium stearate | 1.0 |

| Components | Amounts (% w/w) |
|---|---|
| Solid dispersion of substantially amorphous | 6.3 |
| pimobendan and one or more stabilizing polymers | |
| Lactose monohydrate | 27.0 |
| Microcrystalline cellulose | 29.7 |
| Pork liver powder | 20.0 |
| Starch, pregelatinized | 8.0 |
| Sodium starch glycolate | 6.0 |
| Talc | 2.0 |
| Magnesium stearate | 1.0 |

### EXAMPLES

The following examples serve to further illustrate the present invention; but the same should not be construed as a limitation of the scope of the invention disclosed herein.

### Example 1—Preparation of Solid Dispersion Comprising Amorphous Pimobendan by Hot Melt Extrusion

Crystalline pimobendan (AGC Pharma Chemicals, Spain) and copovidone (BASF Pharma, Germany) were blended in a weight ratio of 10:90. The blend was subsequently processed in a twin-screw extruder. Extrusion was performed at a maximum barrel temperature of 190° C. Extrusion strands obtained were granulated and milled to an average particle size of <500 μm. The substantially amorphous structure of pimobendan in the solid dispersion was characterized by X-ray powder diffraction (see FIG. 1).

### Example 2—Preparation of a Solid Dispersion Comprising Amorphous Pimobendan by Hot Melt Extrusion

Crystalline pimobendan (AGC Pharma Chemicals, Spain) and copovidone (BASF Pharma, Germany) were blended in a weight ratio of 30:70. The blend was subsequently processed in a twin-screw extruder. Extrusion was performed at a maximum barrel temperature of 190° C. Extrusion strands obtained were granulated and milled to an average particle size of <500 μm. The substantially amorphous structure of pimobendan in the solid dispersion was characterized by X-ray powder diffraction (see FIG. 5).

### Example 3—Preparation of a Solid Dispersion Comprising Amorphous Pimobendan by Hot Melt Extrusion

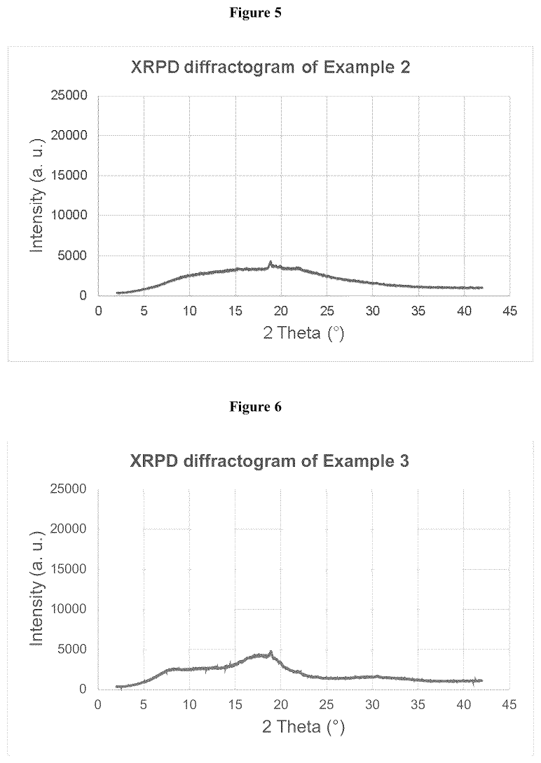

Crystalline pimobendan (AGC Pharma Chemicals, Spain) and Eudragit E PO (Evonik, Germany) were blended in a weight ratio of 5:95. The blend was subsequently processed in a twin-screw extruder. Extrusion was performed at a maximum barrel temperature of 170° C. Extrusion strands obtained were granulated and milled to an average particle size of <500 μm. The substantially amorphous structure of pimobendan in the solid dispersion was characterized by X-ray powder diffraction (see FIG. 6).

### Example 4—Preparation of a Solid Dispersion Comprising Amorphous Pimobendan by Spray Drying

Crystalline pimobendan (AGC Pharma Chemicals, Spain) and povidone (BASF Pharma, Germany) were added to a solvent mixture of methanol and dichloromethane (1:1). The weight ratio of pimobendan and the povidone was 20:80. The resulting clear feed solution was pumped through an atomizer into a drying chamber using a pump at a feed rate of about 400 ml/hour. The solvent was removed to provide a solid dispersion with an average particle size of <100 μm. The substantially amorphous structure of pimobendan in the solid dispersion was characterized by X-ray powder diffraction (see FIG. 1).

### Example 5—Preparation of a Pharmaceutical Composition Comprising the Solid Dispersion According to Example 1

The solid dispersion of amorphous pimobendan obtained according to Example 1 was blended with the excipients listed below in a blender. The resultant mixture was compressed into tablets using a suitable tablet press and suitable punches.

| Components | Amounts (% w/w) |
|---|---|
| Solid dispersion of substantially amorphous | 6.3 |
| pimobendan and one or more stabilizing polymers | |
| Lactose monohydrate | 26.1 |
| Microcrystalline cellulose | 20.6 |
| Pork liver powder | 20.0 |
| Dried yeast | 10.0 |
| Starch, pregelatinized | 8.0 |
| Sodium starch glycolate | 6.0 |
| Talc | 2.0 |
| Magnesium stearate | 1.0 |

### Example 6—Preparation of a Pharmaceutical Composition Comprising the Solid Dispersion According to Example 1

The solid dispersion of amorphous pimobendan obtained according to Example 1 was blended with the excipients listed below in a blender. The resultant mixture was compressed into tablets using a suitable tablet press and suitable punches.

| Components | Amounts (% w/w) |
|---|---|
| Solid dispersion of substantially amorphous | 5.0 |
| pimobendan and one or more stabilizing polymers | |
| Lactose monohydrate | 27.4 |
| Microcrystalline cellulose | 20.6 |
| Pork liver powder | 20.0 |
| Dried yeast | 10.0 |
| Starch, pregelatinized | 8.0 |
| Sodium starch glycolate | 6.0 |
| Talc | 2.0 |
| Magnesium stearate | 1.0 |

### Example 7—Preparation of a Pharmaceutical Composition Comprising the Solid Dispersion According to Example 1

The solid dispersion of amorphous pimobendan obtained according to Example 1 was blended with the excipients and crystalline pimobendan (AGC Pharma Chemicals, Spain, according to FIG. 1) listed below in an appropriate blender. The resultant mixture was compressed into tablets using a suitable tablet press and suitable punches.

| Components | Amounts (% w/w) |
|---|---|
| Solid dispersion of substantially amorphous | 3.1 |
| pimobendan and one or more stabilizing polymers | |
| Pimobendan crystalline | 0.3 |
| Lactose monohydrate | 29.0 |
| Microcrystalline cellulose | 20.6 |
| Pork liver powder | 20.0 |
| Dried yeast | 10.0 |
| Starch, pregelatinized | 8.0 |
| Sodium starch glycolate | 6.0 |
| Talc | 2.0 |
| Magnesium stearate | 1.0 |

### Example 8—Preparation of a Pharmaceutical Composition Comprising the Solid Dispersion According to Example 1

The solid dispersion of amorphous pimobendan obtained according to Example 1 was blended with the excipients and crystalline pimobendan (AGC Pharma Chemicals, Spain, according to FIG. 1) listed below in an appropriate blender. The resultant mixture was compressed into tablets using a suitable tablet press and suitable punches.

| Components | Amounts (% w/w) |
|---|---|
| Solid dispersion of substantially amorphous | 3.1 |
| pimobendan and one or more stabilizing polymers | |
| Pimobendan crystalline | 0.3 |
| Lactose monohydrate | 30.0 |
| Microcrystalline cellulose | 29.6 |
| Pork liver powder | 20.0 |
| Starch, pregelatinized | 8.0 |
| Sodium starch glycolate | 6.0 |
| Talc | 2.0 |
| Magnesium stearate | 1.0 |

### Example 9—XRPD Diffractogram of the Solid Dispersions Prepared According to Examples 1 and 4

XRPD patterns of Examples 1 and 4 (see FIG. 1) were obtained with a PANalytical X′pert Pro X-ray MPD diffractometer using copper K-alpha radiation generated at 40 kV and 40 mA. The XRDP patterns clearly indicate that pimobendan is substantially in amorphous form due to lack of any sharp characteristic peak on the diffractograms of the solid dispersions.

### Example 10—Thermodynamic Solubility

The thermodynamic solubility of crystalline pimobendan and the solid dispersion according to Example 1 was determined at two different pH values representing the gastric juice in dogs at 37° C. by a dynamic dissolution-monitoring instrument using UV calibration, preferably consisting of immersed UV probes connected to the Rainbow instrument (Pion Inc., Billerica MA, USA).

The results obtained are as follows:

Equilibrium solubility (μg/ml)

| | Crystalline | Amorphous |
|---|---|---|
| Buffer pH | pimobendan | pimobendan |
| pH 3.0 (maleic acid buffer) | 19.50 | 79.80 |
| pH 6.5 (phosphate buffer) | 0.30 | 4.41 |

The results clearly indicate that amorphous pimobendan exhibits significantly higher thermodynamic solubility than the crystalline one. In maleic acid buffer at pH 3.0, the difference is about 4-times, whereas in phosphate buffer at pH 6.5 it is more than 10-times.

### Example 11—In Vitro Dissolution Performance

In vitro dissolution profiles of the pharmaceutical compositions according to Example 5 and a commercially available pimobendan tablet formulation were determined at pH 3.0 and pH 6.5. Conditions of testing: instrument similar to USP apparatus 2, V=1000 ml, rotation speed=50 rpm, chromatographic instrument: Agilent Infinity 1290 UHPLC, RP18, 50×3.0 mm, 1.7 μm column.

| | | | | | | |
|---|---|---|---|---|---|---|
| Time (min) | 0 | 10 | 15 | 20 | 30 | 45 |

Dissolution rate (%) - pH 3.0

| | | | | | | |
|---|---|---|---|---|---|---|
| Composition | 0 | 85 | 91 | 92 | 92 | 92 |
| according to | | | | | | |
| Example 5 | | | | | | |
| Commercial | 0 | 64 | 81 | 91 | 93 | 93 |
| formulation | | | | | | |

Dissolution rate (%) - pH 6.5

| | | | | | | |
|---|---|---|---|---|---|---|
| Composition | 0 | 55 | 66 | 70 | 75 | 77 |
| according to | | | | | | |
| Example 5 | | | | | | |
| Commercial | 0 | 44 | 62 | 72 | 78 | 83 |
| formulation | | | | | | |

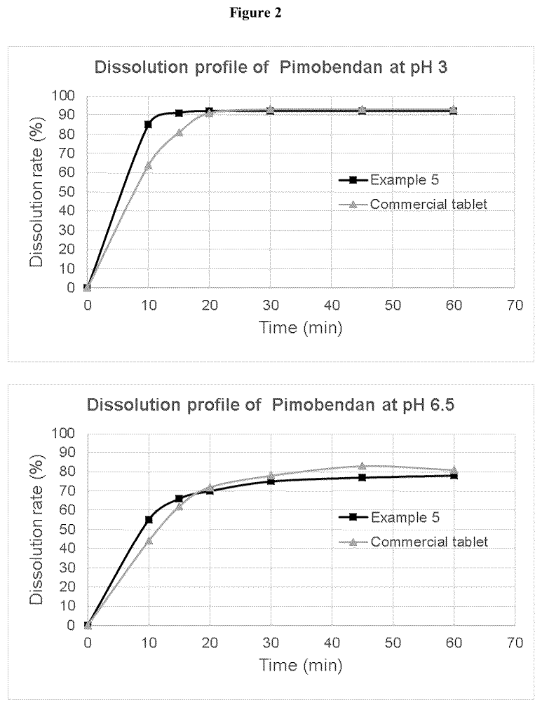

In 1000 mL of dissolving medium, the dissolution rate of the pharmaceutical composition comprising the solid dispersion according to Example 1 was comparable to that of the commercial formulation at both pH values representing the gastric juice (see FIG. 2).

### Example 12—Biorelevant Volume Dissolution and Permeation

Biorelevant volume dissolution and permeation of the pharmaceutical compositions according to Examples 5 and 7, and a commercially available pimobendan tablet formulation were determined at default and high pH conditions. Default conditions: pH 3.0 representing the stomach for 30 minutes, then pH 6.2 representing the small intestines up to 120 minutes in the donor chamber, and pH 7.4 representing the blood in the acceptor chamber. High pH conditions: pH 6.5 representing the stomach for 30 minutes, then, pH 7.5 representing the small intestines up to 120 minutes in the donor chamber, and pH 7.4 characterizing the blood in the acceptor chamber. Pimobendan concentrations were determined by immersed UV probes, such as by using the μFLUX apparatus (Pion Inc., Billerica MA, USA). In vitro flux was determined from the concentration-time profiles in the acceptor chamber recorded in the interval of 60-120 minutes. Permeability was calculated from the flux value and the drug substance concentration in the donor chamber.

Amounts of pimobendan (drug) dissolved and then permeated through the membrane were determined as follows:

| | pH conditions | | | | | |
|---|---|---|---|---|---|---|
| | Donor chamber: pH 3.0-6.2, acceptor chamber: 7.4 | | | | | |
| | Drug concentrations | | | | | |
| | Amount dissolved (μg/ml) | | | Amount permeated (μg/ml) | | |
| Time (min) | 0 | 15 | 30 | 60 | 90 | 120 |
| Composition acc. | 0 | 71 | 73 | 0.80 | 1.39 | 1.95 |
| Example 5 | | | | | | |
| Composition acc. | 0 | 61 | 62 | 0.67 | 1.12 | 1.56 |
| Example 7 | | | | | | |
| Commercial | 0 | 65 | 64 | 0.35 | 0.66 | 0.97 |
| formulation | | | | | | |
| | pH conditions | | | | | |
| | Donor chamber: pH 6.5-7.5, acceptor chamber 7.4 | | | | | |
| | Drug concentrations | | | | | |
| | Amount dissolved (μg/ml) | | | Amount permeated (μg/ml) | | |
| Time (min) | 0 | 15 | 30 | 60 | 90 | 120 |
| Composition acc. | 0 | 83 | 81 | 1.33 | 1.92 | 2.50 |
| Example 5 | | | | | | |
| Composition acc. | 0 | 38 | 37 | 0.82 | 1.23 | 1.63 |
| Example 7 | | | | | | |
| Commercial | 0 | 48 | 48 | 0.47 | 0.80 | 1.13 |
| formulation | | | | | | |

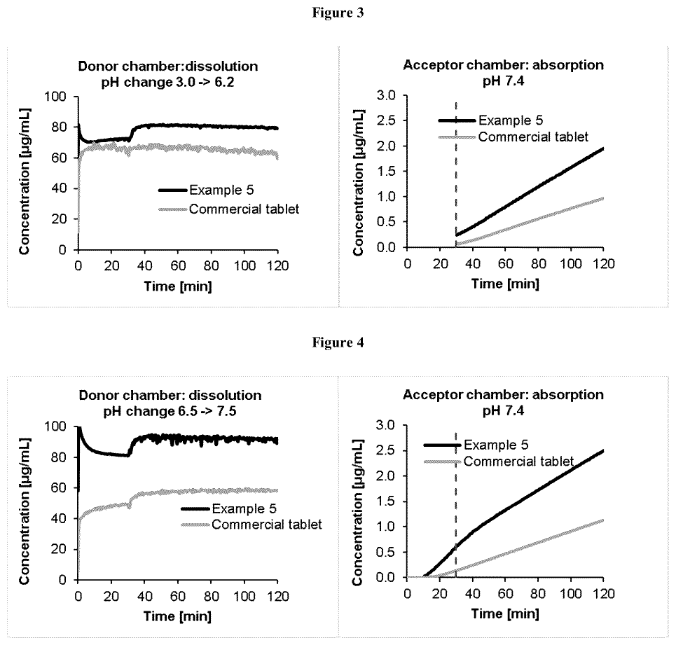

At both default and high pH conditions, the amounts dissolved from the pharmaceutical composition containing the solid dispersions comprising substantially amorphous pimobendan was higher than from the commercial formulation. The amounts permeated exhibited even a higher difference (see FIGS. 3 and 4, respectively).

In vitro transmembrane flux values characterizing the total amount of material crossing a unit area of the biological membrane per unit time and thus the rate of absorption were calculated as follows:

| | In vitro flux [μg/(min * cm²⁾] | | | |
|---|---|---|---|---|
| | Donor | | Donor | |
| | pH | Acceptor | pH | Acceptor |
| Formulation | 3.0-6.2 | pH 7.4 | 6.5-7.5 | pH 7.4 |

| | | |
|---|---|---|
| Composition acc. Example 5 | 0.250 | 0.242 |
| Composition acc. Example 7 | 0.191 | 0.175 |
| Commercial formulation | 0.133 | 0.144 |

The calculated in vitro flux values for the pharmaceutical compositions comprising the solid dispersions comprising substantially amorphous pimobendan were much higher than that for the commercial formulation, thus predicting an improved bioavailability of the solid dispersions/pharmaceutical compositions of the present invention. The improved bioavailability is due to the higher solubility, dissolution, supersaturation and permeation of amorphous pimobendan compared to the commercial formulation.

### REFERENCES

(1) CN 112 618 505
(2) EP 0008 391
(3) EP 0 439 030
(4) EP 2 338 493
(5) Rekis, T. et al., Cryst. Growth Des. 2018, 18(1): 264-273
(6) Vasconcelos T et al., Drug Discovery Today 2012, 12: 1068-1075
(7) WO 2005/084647
(8) WO 2008/055871
(9) WO 2010/010257
(10) WO 2010/055119
(11) WO 2011/042463
(12) WO 2015/082389
(13) WO 2017/103054
(14) WO 2021/081366

The following clauses are also part of the present invention and general disclosure:
1. A solid dispersion comprising, preferably consisting of, amorphous pimobendan, preferably substantially amorphous pimobendan, and one or more stabilizing polymers.
2. The solid dispersion according to clause 1, wherein the one or more stabilizing polymers is selected from the group consisting of: polymerization products of N-vinylpyrrolidone and vinylpyrrolidone-vinyl acetate as well as polymeric methacrylates.
3. The solid dispersion according to clause 2, wherein the polymerization products of N-vinylpyrrolidone and vinylpyrrolidone-vinyl acetate are selected from the group consisting of: povidone (polyvinylpyrrolidone) and copovidone [poly-(1-vinylpyrrolidone-co-vinylacetate)]; and the polymeric methacrylates are selected from the group consisting of: polymeric buyl 2-methylprop-2-enoate; polymeric 2-(dimethylamino)ethyl 2-methylprop-2-enoate; and polymeric methyl 2-methylprop-2-enoate.
4. The solid dispersion according to any one of clauses 1 to 3, wherein the one or more stabilizing polymers is copovidone [poly-(1-vinylpyrrolidone-co-vinylacetate)], which is preferably the only stabilizing polymer in the solid dispersion.
5. The solid dispersion according to any one of clauses 1 to 4, wherein pimobendan is present in an amount of 1% to 80% by weight relative to the weight of the solid dispersion, preferably in an amount of 5% to 40% by weight relative to the weight of the solid dispersion and more preferably in an amount of 10% to 20% by weight relative to the weight of the solid dispersion.
6. The solid dispersion according to any one of clauses 1 to 4, wherein at least 70% of the pimobendan is present in amorphous form.
7. A process of preparing the solid dispersion according to any one of clauses 1 to 6, wherein the solid dispersion is prepared by a melting-based process, preferably by hot melt extrusion, or a solvent evaporation-based process, preferably by spray drying.
8. The process according to clause 7 being a hot melt extrusion process comprising the steps of:
   (a) processing pimobendan and the one or more stabilizing polymers by means of a pharmaceutical extruder, preferably a screw extruder, more preferably a twin-screw extruder, preferably at a barrel temperature of 120-200° C., more preferably at a barrel temperature of 150-180° C., to obtain an extrudate,
   (b) comminuting the extrudate obtained in step (a) by using a granulator or pelletizer to obtain a granulate, and
   (c) milling the granulate obtained in step (b) to obtain a solid dispersion, the solid dispersion preferably comprising particles with an average diameter of less than 500 μm.
9. The process according to clause 7 being a spray drying process comprising the steps of:
   (a) dispersing or dissolving pimobendan and the one or more stabilizing polymers in one or more solvents, preferably selected from the group consisting of: dichloromethane, chloroform, ethanol, methanol, 2-propanol, ethyl acetate, acetone, water or mixtures thereof, to obtain a feed solution,
   (b) pumping the feed solution obtained in step (a) through an atomizer into a drying chamber, and
   (c) removing the one or more solvents in the drying chamber to obtain a solid dispersion, the solid dispersion preferably comprising particles with an average diameter of less than 500 μm.
10. A solid dispersion comprising, preferably consisting of, amorphous pimobendan, preferably substantially amorphous pimobendan, and one or more stabilizing polymers obtainable by the process according to any one of clauses 7 to 9.
11. A pharmaceutical composition comprising the solid dispersion according to any one of clauses 1 to 6 and 10 and one or more pharmaceutically acceptable excipients, optionally additionally comprising crystalline form(s) of pimobendan (preferably according to FIG. 1), wherein preferably the pharmaceutical composition is a tablet, more preferably a chewable tablet.
12. The pharmaceutical composition according to clauses 11, wherein the pharmaceutically acceptable excipients comprise at least one filler, at least one disintegrant, at least one lubricant and at least one flavor.
13. The pharmaceutical composition according to any one of clauses 11 to 12, further comprising pharmaceutically effective amounts of one or more further active ingredients selected from the groups of angiotensin converting enzyme (ACE) inhibitors, aldosterone antagonists and/or loop diuretics.
14. The pharmaceutical composition according to clause 13, wherein the optional one or more further active ingredients selected from the groups of angiotensin converting enzyme (ACE) inhibitors, aldosterone antagonists and/or loop diuretics are independently from each other benazepril, spironolactone, furosemide and/or derivatives thereof, in free form or in the form of a physiologically acceptable salt.
15. The pharmaceutical composition according to any one of clauses 11 to 14 selected from the group consisting of: A, B, C, D:

| Components | Amounts (% w/w) |
|---|---|
| Solid dispersion of substantially amorphous | 6.3 |
| pimobendan and one or more stabilizing polymers | |
| Lactose monohydrate | 26.1 |
| Microcrystalline cellulose | 20.6 |
| Pork liver powder | 20.0 |
| Dried yeast | 10.0 |
| Starch, pregelatinized | 8.0 |
| Sodium starch glycolate | 6.0 |
| Talc | 2.0 |
| Magnesium stearate | 1.0 |

| Components | Amounts (% w/w) |
|---|---|
| Solid dispersion of substantially amorphous | 5.0 |
| pimobendan and one or more stabilizing polymers | |
| Lactose monohydrate | 27.4 |
| Microcrystalline cellulose | 20.6 |
| Pork liver powder | 20.0 |
| Dried yeast | 10.0 |
| Starch, pregelatinized | 8.0 |
| Sodium starch glycolate | 6.0 |
| Talc | 2.0 |
| Magnesium stearate | 1.0 |

| Components | Amounts (% w/w) |
|---|---|
| Solid dispersion of substantially amorphous | 3.1 |
| pimobendan and one or more stabilizing polymers | |
| Pimobendan crystalline | 0.3 |
| Lactose monohydrate | 29.0 |
| Microcrystalline cellulose | 20.6 |
| Pork liver powder | 20.0 |
| Dried yeast | 10.0 |
| Starch, pregelatinized | 8.0 |
| Sodium starch glycolate | 6.0 |
| Talc | 2.0 |
| Magnesium stearate | 1.0 |

| Components | Amounts (% w/w) |
|---|---|
| Solid dispersion of substantially amorphous | 3.1 |
| pimobendan and one or more stabilizing polymers | |
| Pimobendan crystalline | 0.3 |
| Lactose monohydrate | 30.0 |
| Microcrystalline cellulose | 29.6 |
| Pork liver powder | 20.0 |
| Starch, pregelatinized | 8.0 |
| Sodium starch glycolate | 6.0 |
| Talc | 2.0 |
| Magnesium stearate | 1.0 |

1. A solid dispersion comprising, preferably consisting of, amorphous pimobendan, preferably substantially amorphous pimobendan, and one or more stabilizing polymers.

2. The solid dispersion according to claim 1, wherein the one or more stabilizing polymers is selected from the group consisting of: polymerization products of N-vinylpyrrolidone, vinyl acetate and vinylpyrrolidone mixed with vinyl acetate; as well as polymeric methacrylates.

3. The solid dispersion according to claim 2, wherein the polymerization products of N-vinylpyrrolidone, vinyl acetate and vinylpyrrolidone mixed with vinyl acetate; as well as polymeric methacrylates are selected from the group consisting of: povidone (polyvinylpyrrolidone), polyvinyl alcohol and copovidone [poly-(1-vinylpyrrolidone-co-vinylacetate)]; and the polymeric methacrylates are selected from the group consisting of: polymeric butyl 2-methylprop-2-enoate; polymeric 2-(dimethylamino)ethyl 2-methylprop-2-enoate; and polymeric methyl 2-methylprop-2-enoate.

4. The solid dispersion according to any one of claims 1 to 3, wherein the solid dispersion; and/or the one or more stabilizing polymers; and/or the polymerization products of N-vinylpyrrolidone, vinyl acetate and vinylpyrrolidone mixed with vinyl acetate; and/or polymeric methacrylates independently from each other do not comprise crospovidone (cross-linked polyvinylpyrrolidone, polyvinylpolypyrrolidone, PVPP).

5. The solid dispersion according to any one of claims 1 to 4, wherein the solid dispersion; and/or the one or more stabilizing polymers; and/or the polymerization products of N-vinylpyrrolidone, vinyl acetate and vinylpyrrolidone mixed with vinyl acetate; and/or polymeric methacrylates independently from each other do not comprise one or more hyperbranched polymers, optionally being selected from the group consisting of: dendritic polymers, dendrimers, arborol, cascade, cauliflower or star polymers, polydisperse hyperbranched polymers, dendrigraft polymers, or other high molecular weight polymers, which all have specific branched structure containing a center atom or a molecule, which can be monomeric or polymeric, from where three or more chains emanate; hyperbranched polyimines, hyperbranched polyurethanes, hyperbranched polyamides, hyperbranched polyesteramines, hyperbranched polyesteramides, hyperbranched polymers comprising hydroxyl groups, ester groups, amido groups and/or carboxyl groups, and polyesteramide hyperbranched polymers, such as one or more hyperbranched polyesteramide having tertiary amine end groups and/or having hydroxyl end groups.

6. The solid dispersion according to any one of claims 1 to 5, wherein the one or more stabilizing polymers is copovidone [poly-(1-vinylpyrrolidone-co-vinylacetate)], which is preferably the only stabilizing polymer in the solid dispersion.

7. The solid dispersion according to any one of claims 1 to 6, wherein pimobendan is present in an amount of 1% to 80% by weight relative to the weight of the solid dispersion, preferably in an amount of 5% to 40% by weight relative to the weight of the solid dispersion and more preferably in an amount of 10% to 20% by weight relative to the weight of the solid dispersion.

8. The solid dispersion according to any one of claims 1 to 7, wherein at least 70% of the pimobendan is present in amorphous form.

9. A process of preparing the solid dispersion according to any one of claims 1 to 8, wherein the solid dispersion is prepared by a melting-based process, preferably by hot melt extrusion, or a solvent evaporation-based process, preferably by spray drying.

10. The process according to claim 9 being a hot melt extrusion process comprising the steps of:
(a) processing pimobendan and the one or more stabilizing polymers by means of a pharmaceutical extruder, preferably a screw extruder, more preferably a twin-screw extruder, preferably at a barrel temperature of 120-200° C., more preferably at a barrel temperature of 150-180° C., to obtain an extrudate,
(b) comminuting the extrudate obtained in step (a) by using a granulator or pelletizer to obtain a granulate, and
(c) milling the granulate obtained in step (b) to obtain a solid dispersion, the solid dispersion preferably comprising particles with an average diameter of less than 500 μm.

11. The process according to claim 9 being a spray drying process comprising the steps of:
(a) dispersing or dissolving pimobendan and the one or more stabilizing polymers in one or more solvents, preferably selected from the group consisting of: dichloromethane, chloroform, ethanol, methanol, 2-propanol, ethyl acetate, acetone, water and mixtures thereof, to obtain a feed solution,
(b) pumping the feed solution obtained in step (a) through an atomizer into a drying chamber, and
(c) removing the one or more solvents in the drying chamber to obtain a solid dispersion, the solid dispersion preferably comprising particles with an average diameter of less than 500 μm.

12. A solid dispersion comprising, preferably consisting of, amorphous pimobendan, preferably substantially amorphous pimobendan, and one or more stabilizing polymers obtainable by the process according to any one of claims 9 to 11.

13. A pharmaceutical composition comprising the solid dispersion according to any one of claims 1 to 8 and 12 and one or more pharmaceutically acceptable excipients, optionally additionally comprising crystalline form(s) of pimobendan (preferably according to FIG. 1), wherein preferably the pharmaceutical composition is a tablet, more preferably a chewable tablet.

14. The pharmaceutical composition according to claim 13, wherein the pharmaceutically acceptable excipients comprise at least one filler, at least one disintegrant, at least one lubricant and at least one flavor.

15. The pharmaceutical composition according to any one of claims 13 to 14, further comprising pharmaceutically effective amounts of one or more further active ingredients selected from the groups of angiotensin converting enzyme (ACE) inhibitors, aldosterone antagonists and/or loop diuretics.

16. The pharmaceutical composition according to claim 15, wherein the optional one or more further active ingredients selected from the groups of angiotensin converting enzyme (ACE) inhibitors, aldosterone antagonists and/or loop diuretics are independently from each other benazepril, spirolactone, furosemide aid/or derivatives thereof, in free form or in the form of a physiologically acceptable salt.

17. The pharmaceutical composition according to anyone of claims 11 to 14 selected from the group consisting of: A, B, C, D and E (the crystalline pimobendan depicted in the tables below preferably refers to the crystalline pimobendan according to FIG. 1):

| Components | Amounts (% w/w) |
|---|---|
| Solid dispersion of substantially amorphous | 6.3 |
| pimobendan and one or more stabilizing polymers | |
| Lactose monohydrate | 26.1 |
| Microcrystalline cellulose | 20.6 |
| Pork liver powder | 20.0 |
| Dried yeast | 10.0 |
| Starch, pregelatinized | 8.0 |
| Sodium starch glycolate | 6.0 |
| Talc | 2.0 |
| Magnesium stearate | 1.0 |
| Components | Amounts (% w/w) |
|---|---|
| Solid dispersion of substantially amorphous | 5.0 |
| pimobendan and one or more stabilizing polymers | |
| Lactose monohydrate | 27.4 |
| Microcrystalline cellulose | 20.6 |
| Pork liver powder | 20.0 |
| Dried yeast | 10.0 |
| Starch, pregelatinized | 8.0 |
| Sodium starch glycolate | 6.0 |
| Talc | 2.0 |
| Magnesium stearate | 1.0 |
| Components | Amounts (% w/w) |
|---|---|
| Solid dispersion of substantially amorphous | 3.1 |
| pimobendan and one or more stabilizing polymers | |
| Pimobendan crystalline | 0.3 |
| Lactose monohydrate | 29.0 |
| Microcrystalline cellulose | 20.6 |
| Pork liver powder | 20.0 |
| Dried yeast | 10.0 |
| Starch, pregelatinized | 8.0 |
| Sodium starch glycolate | 6.0 |
| Talc | 2.0 |
| Magnesium stearate | 1.0 |
| Components | Amounts (% w/w) |
|---|---|
| Solid dispersion of substantially amorphous | 3.1 |
| pimobendan and one or more stabilizing polymers | |
| Pimobendan crystalline | 0.3 |
| Lactose monohydrate | 30.0 |
| Microcrystalline cellulose | 29.6 |
| Pork liver powder | 20.0 |
| Starch, pregelatinized | 8.0 |
| Sodium starch glycolate | 6.0 |
| Talc | 2.0 |
| Magnesium stearate | 1.0 |
| Components | Amounts (% w/w) |
|---|---|
| Solid dispersion of substantially amorphous | 6.3 |
| pimobendan and one or more stabilizing polymers | |
| Lactose monohydrate | 27.0 |
| Microcrystalline cellulose | 29.7 |
| Pork liver powder | 20.0 |
| Starch, pregelatinized | 8.0 |
| Sodium starch glycolate | 6.0 |
| Talc | 2.0 |
| Magnesium stearate | 1.0 |
